(19) United States
(12) Patent Application Publication
(10) Pub. No.: US 2025/0255314 A1
(43) Pub. Date: Aug. 14, 2025

(54) Enzymatic Modification of Legume Rich Doughs to Produce Machinable Sheeted Snack Products

(71) Applicant: Frito-Lay North America, Inc., Plano, TX (US)

(72) Inventors: Antonio Garay, The Colony, TX (US); Yi Zhu, Plano, TX (US); Sathya B. Kalambur, Lewisville, TX (US)

(21) Appl. No.: 18/891,551

(22) Filed: Sep. 20, 2024

Related U.S. Application Data
(63) Continuation-in-part of application No. 18/441,572, filed on Feb. 14, 2024.

(51) Int. Cl. A21D 13/045; A21D 8/04; A21D 10/00; C12N 9/10

(52) U.S. Cl. CPC A21D 13/045; A21D 8/042; A21D 10/002; C12N 9/1044; C12Y 203/02013

(57) ABSTRACT

A snack product includes legume, deactivated transglutaminase (TG), oil, moisture, and/or seasonings. The legume dough used to make the snack product includes transglutaminase (TG) to form a modified dough having improved sheetability characteristics as compared to a similar dough without TG. The legume dough may include from about 60 wt. % to about 100 wt. % legumes. An exemplary method of making the modified legume dough includes adding about 0.5 wt. % to about 5 wt. % TG, incubating the modified legume dough at about 100° F. to about 140° F. for between about 5 minutes to about 120 minutes.

### CROSS-REFERENCE TO RELATED APPLICATIONS

This patent application is a continuation-in-part of application Ser. No. 18/441,572, filed Feb. 14, 2024, the entire contents of which are incorporated herein by reference.

### TECHNICAL FIELD OF THE INVENTION

Methods of making a legume based snack product and a dough for forming the legume based snack product are described. The legume based snack product is made from dough that contains at least about 60 wt. % legume or pulse, e.g., chickpea.

### BACKGROUND

Pulses have earned increased attention both in the scientific and the food industry communities due to their nutritional make-up, health benefits, wide availability at scale with affordable pricing, their positive impact in food sustainability, and their versatility in kitchen applications. The term pulse refers to the dry seeds obtained from leguminous crops with oil contents typically below 10% on dry basis and consumed without their pods. Examples of pulses include chickpeas, lentils, peas, beans, lupins, vetches, among others.

Chickpeas are a type of legume that has received increased attention in recent years due to its advantaged nutritional profile, and relatively low cost. Incorporating pulses, such as chickpea, into snack products such as tortilla-style chips could enhance nutrition value and consumer health perception. However, legumes like chickpea have different kernel properties and contain different amounts and types of proteins, starches, fats, and fibers compared to corn (which is the typical main ingredient of tortillas), which lead to process and product challenges. From a dough perspective, replacing a major amount of corn with pulses results in sticky/stiff doughs with low cohesiveness and poor sheetability. Finished snack products with high levels of pulses may also display dry mouthfeel, chalky after-taste, and gritty texture. Effects of legume inclusion are largely dependent on the physicochemical and functional properties of legumes, such as protein solubility, water holding capacity (WHC), pasting property, etc. Protein solubility plays a crucial role in influencing other functional properties of flours; hence, the various applications of legume flours.

A challenge to producing snack products that contain legumes is related to the processing of the dough comprising the legume. Challenges include low dough cohesiveness, high stickiness, and poor dough sheetability. Thus, there is a need to improve the dough performance when producing the legume based snack products, where the dough comprises about 60 wt. % to about 100 wt. % legumes.

### SUMMARY

In one aspect, a dough comprising about 60 wt. % to about 100 wt. % legume, and transglutaminase (TG) is described. In other aspects, the transglutaminase may be a low oxidation transglutaminase. The dough may be used to make a snack product. The legume used to make the dough may be any known legume such as, but not limited to, peas, grass peas, green peas, black-eyed peas, broad beans, chickpeas, lentils, lupins, mesquite, carob, tamarind, alfalfa, clover, soybeans, lima beans, black beans, kidney beans, navy beans, butter beans, adzuki beans, peanuts, or pinto beans, and mixtures thereof. The dough may contain about 0.5 wt. % to about 5 wt. % transglutaminase or, when low oxidation transglutaminase is used, the dough may contain about 0.5 wt. % to about 5 wt. % low oxidation transglutaminase. The dough may be made from legume kernels or from raw legume flour. Before being used to form a sheet, the dough may contain a solubility of less than about 20 grams per 100 grams of the dough.

A snack product may be produced by machining the dough to form a sheet. The sheet may have a thickness of about 0.5 mm to about 3.0 mm. It is contemplated that the sheet may be cut into raw snack products (e.g., raw chips) and that the raw snack products may be dehydrated by frying or baking to form the snack product. Upon dehydrating, the snack product may have an oil uptake or absorption of less than about 30 wt. %. The snack product may be a tortilla-style chip or a similar snack product.

In another aspect, a snack product that includes about 40 wt. % to about 100 wt. % of a legume, deactivated transglutaminase (or deactivated low oxidation transglutaminase), and less than about 30 wt. % oil is described. The legume in the snack product the legume may be any known legume such as, but not limited to, peas, grass peas, green peas, black-eyed peas, broad beans, chickpeas, lentils, lupins, mesquite, carob, tamarind, alfalfa, clover, soybeans, lima beans, black beans, kidney beans, navy beans, butter beans, adzuki beans, peanuts, or pinto beans, and mixtures thereof. The snack product may comprise about 22 wt. % to about 26 wt. % oil, including any percentage or range comprised therein. The snack product may further comprise about 10% seasoning.

In another aspect, a method of reducing the stickiness of a legume dough is described. The method may comprise adding about 0.05 wt. % to about 5 wt. % transglutaminase (or low oxidation transglutaminase) to the dough; wherein the dough comprises between about 60 wt. % to about 100 wt. % legume, and holding the dough at a temperature between about 100° F. to about 140° F. for between about 5 minutes to about 120 minutes.

The transglutaminase (or low oxidation transglutaminase) may crosslink the protein present in legumes, including the soluble globulin and albumin fractions found in the legume dough water trapping within the aggregated protein matrix, and reducing the stickiness of the legume dough.

The method may be used to make doughs that include legumes such as, but not limited to, peas, grass peas, green peas, black-eyed peas, broad beans, chickpeas, lentils, lupins, mesquite, carob, tamarind, alfalfa, clover, soybeans, lima beans, black beans, kidney beans, navy beans, butter beans, adzuki beans, peanuts, or pinto beans, and mixtures thereof.

The method may be implemented when legume dough is used to make legume rich snacks with higher protein content compared to those done with cereals like corn. The snack products made by implementing this method may have an oil uptake of less than about 30 wt. % through frying and/or baking dehydration methods. The snack products made by implementing this method may comprise about 10 wt. % seasonings.

For the ease of description, the described doughs, snack products, and methods refer to chickpea. In this regard, it should be understood that the reference to chickpea applies to all legumes (including those noted above), except where it is evident that in the specific context, the reference is solely to chickpea, e.g., some described formulas.

All percentages recited above and in the following description and claims are percent by weight unless specifically noted otherwise. Other aspects and advantages of this invention will be appreciated from the following detailed description.

### BRIEF DESCRIPTION OF THE DRAWINGS

FIG. 1 illustrates one method of preparing a modified legume dough according to the described embodiments, where chickpea is used as an exemplary legume.

FIG. 2 is a graph showing the impact of different amounts of transglutaminase (TG) on dough adhesiveness.

FIG. 3A illustrates an instance when a comparative dough (i.e., not according to the exemplary described doughs) used to form a sheet results in shreds exiting a cutting machine because the dough possesses a high level of stickiness.

FIG. 3B illustrates an instance when a dough according to the exemplary described doughs used to form a sheet results in cleanly cut raw chips exiting a cutting machine.

FIG. 4A illustrates a sheet of dough used to form a snack product having a sheeting score of 1.

FIG. 4B illustrates a sheet of dough used to form a snack product having a sheeting score of 2.

FIG. 4C illustrates a sheet of dough used to form a snack product having a sheeting score of 3.

FIG. 4D illustrates a sheet of dough used to form a snack product having a sheeting score of 4.

FIG. 5A illustrates sheeting scores of doughs having no corn as a function of different incubation temperatures and different transglutaminase (TG) amounts.

FIG. 5B illustrates sheeting scores of dough comprising legume and corn as a function of different incubation temperatures and different transglutaminase (TG) amounts.

### DETAILED DESCRIPTION OF THE INVENTION

In some aspects, legumes including, but not limited to, chickpeas, lentils, and/or pinto beans may be used to produce snack products. In some embodiments, legumes may be in the form of whole seeds, whole cooked seeds, partially or fully dehulled or decorticated seeds, partially or fully dehulled or decorticated cooked seeds, partially or fully de-oiled seeds, raw whole grits, raw partially or fully dehulled or decorticated grits, partially or fully de-oiled grits, raw whole flours, raw partially or fully dehulled/decorticated flours, raw partially or fully de-oiled flours, raw protein fractions, readily soluble legume protein, or any mixtures thereof. As used in this description, the word “legume” refers to all forms of legumes including, but not limited to those noted above, unless the specific context notes otherwise.

In one embodiment, a legume dough (e.g., chickpea dough) may be used to make the snack products. Whole legumes, native legume flour, or raw legume flour may be used to make the legume dough. The legume dough may be by mixing the legume with water at a temperature that ranges from about room temperature (e.g., about 65° F.) to about 212° F. In some aspects, the physicochemical and structural properties of the legume dough may be modified to enhance legume dough performance when making a snack product. In some embodiments, the snack product may be chips (e.g., tortilla-style chips) or some other sheeted fried or baked snack.

A tortilla-style chip platform typically involves the creation of a corn-based dough or “masa” from nixtamalized corn to produce tortilla chips (TC), tortillas, corn chips, corn-based snacks, and other snack products. In a typical nixtamalization process, the cooked corn may be steeped for several hours in a solution of calcium hydroxide (Ca(OH)₂) at pH 11. The steeped corn may then washed with additional water to reduce the pH to neutrality and to remove nejayote (the calcium hydroxide-rich liquors resulting from the steeping step) to form clean corn. The clean corn may be stone-milled into a soft non-sticky masa. The calcium hydroxide used in the nixtamalization process is often referred to as lime. During this process, several critical physicochemical and structural changes take place, such as softening and solubilization of pericarp tissue of the corn, diffusion, and absorption of water and calcium ions into the corn kernel's endosperm, full hydration of starch and protein molecules, partial gelatinization of starch, and denaturation/polymerization of proteins. These changes are responsible for the smooth texture and sensory properties of masa and the corresponding snack products. However, the use of legumes in addition to or instead of corn to make a legume dough increases the stickiness of the resultant dough and affects dough sheetability. Sheetability refers to the ability to form sheets from the dough during the snack making process.

Cooking the legume at different conditions (e.g., high temperatures) or adding acid may potentially positively affect legume dough properties and enhance sheetability. Cooking the legume to high temperatures may reduce protein solubility, increase water holding capacity, and/or alter the pasting properties of the legume dough, thus enhancing the legume dough performance when incorporated into the snack products. However, cooking the legume to high temperatures such as 160° F. to about 205° F. also increases starch gelatinization, thereby increasing oil pick-up in the resulting snack product. Oil pick-up refers to the amount of oil in the final snack product, which may occur as a result of frying the dough to form the snack product. Similarly, while adding acid to the legume dough may improve sheeting behavior, adding acid makes the snack product very sour, and therefore inedible. The following describes different methods to increase the sheetability of legume dough while generating healthy and edible snack products.

With the above in mind, it is contemplated that the described doughs and methods of making the doughs will be free of cooking to the above high temperatures, i.e., the doughs will cooked at temperatures from about 125° F. to about 145° F. and will be free of any added acid, and the described doughs and methods of making the doughs will have minimal (or no) amount of gelatinized starches.

### Modified Legume Dough

In some aspects, a modified legume dough is used to prepare the snack product. In some embodiments, the modified legume dough used to prepare the snack product comprises, consists essentially of, or consists of a mixture of legume dough, and transglutaminase (TG). In some embodiments, the TG may be obtained commercially from Ajinimoto Health 7 Nutrition North America, Inc under the brand name ACTIVA®, including but not limited to ACTIVA® T1.

In other embodiments, the modified legume dough used to prepare the snack product comprises, consists essentially of, or consists of a mixture of legume dough, and low oxidation transglutaminase (TG). Low oxidation TG refers to a TG preparation that, includes other components to maintain the TG catalytic activity and to protect the TG against oxidation when used in the presence of oxygen in environments such as those when air is present. As an example, the low oxidation TG may be formed by coating the TG with maltodextrin, calcium chloride, or a mixture of maltodextrin and calcium chloride. In some embodiments, low oxidation TG may be commercially obtained from Ajinimoto Health 7 Nutrition North America, Inc under the brand name ACTIVA@STG-T1.

In some aspects, the legume dough used to prepare the modified legume dough comprises, consists essentially of, or consists of a mixture of legume and nixtamalized corn. The amount of legume in the legume dough may be about 60 wt. % to about 65 wt. %, about 65 wt. % to about 70 wt. %, about 70 wt. % to about 75 wt. %, about 75 wt. % to about 80 wt. %, about 80 wt. % to about 95 wt. %, about 85 wt. % to about 90 wt. %, about 90 wt. % to about 95 wt. %, about 95 wt. % to about 100 wt. %, including any percentage or range comprised therein. In some embodiments, the amount of legume in the legume dough may be in the range of about 60 wt. % to about 100 wt. %, including any percentage comprised therein. The legume dough may contain about 2 wt. % to about 10 wt. % legume hull, including any percentage or range comprised therein. For example, legume dough made with about 100 wt. % kabuli chickpea may contain about 2.5 wt. % to about 3 wt. % kabuli chickpea hull. Legume dough made with about 100 wt. % desi chickpea may comprise about 7 wt. % to about 7.7 wt. % desi chickpea hull. In some embodiments, the legume dough may further comprise flavoring, antioxidant, and/or other functional agent.

In those instances where the legume is chickpea, the chickpea can be provided as raw chickpea flour, native chickpea flour, or a combination of both that is hydrated or whole chickpea that has been ground to produce a chickpea flour. In this regard, it has been found that fully cooked chickpea flour is not suitable for forming the desired dough or the snack product because fully cooked chickpea flour contains denatured proteins and gelatinized starch, the latter of which increases the amount of oil pickup upon frying. Accordingly, it is contemplated that the described doughs, products, and methods of making such are free of fully cooked chickpea flour.

The amount of nixtamalized corn may be about 0 wt. % to about 5 wt. %, about 5 wt. % to about 10 wt. %, about 10 wt. % to about 15 wt. %, about 15 wt. % to about 20 wt. %, about 20 wt. % to about 25 wt. %, about 25 wt. % to about 30 wt. %, about 30 wt. % to about 35 wt. %, about 35 wt. % to about 40 wt. %, including any percentage comprised therein.

The amount of TG in the modified legume dough may be about 0.3 wt. % to about 0.5 wt. %, about 0.5 wt. % to about 0.8 wt. %, about 0.8 wt. % to about 1.0 wt. %, about 1.0 wt. % to about 1.4 wt. about 1.4 wt. % to about 1.8 wt. %, about 1.8 wt. % to about 2.0 wt. %, about 2.0 wt. % to about 2.5 wt. %, about 2.5 wt. % to about 3.0 wt. %, about 3.0 wt. % to about 3.5 wt. %, about 3.5 wt. % to about 4.0 wt. %, about 4.0 wt. % to about 4.5 wt. %, or about 4.5 wt. % to about 5.0 wt. %, including any percentage or range comprised therein.

The amount of low oxidation TG in the modified legume dough may be about 0.3 wt. % to about 0.5 wt. %, about 0.5 wt. % to about 0.8 wt. %, about 0.8 wt. % to about 1.0 wt. %, about 1.0 wt. % to about 1.4 wt. about 1.4 wt. % to about 1.8 wt. %, about 1.8 wt. % to about 2.0 wt. %, about 2.0 wt. % to about 2.5 wt. %, about 2.5 wt. % to about 3.0 wt. %, about 3.0 wt. % to about 3.5 wt. %, about 3.5 wt. % to about 4.0 wt. %, about 4.0 wt. % to about 4.5 wt. %, or about 4.5 wt. % to about 5.0 wt. %, including any percentage or range comprised therein.

In some embodiments, the modified legume dough may comprise asparaginase (AG). In some embodiments, the modified legume dough may not comprise asparaginase (AG). In other words, the modified legume dough is free of asparaginase.

In some embodiments, the modified legume dough may contain about 48 wt. % to about 50 wt. %, about 50 wt. % to about 52 wt. %, about 52 wt. % to about 54 wt. %, about 54 wt. % to about 56 wt. %, or about 56 wt. % to about 58 wt. % moisture or water, including any percentage or range comprised therein. In some embodiments, the modified legume dough may contain moisture in a range where the lower limit may be from any one of 52.5 wt. % or about 52 wt. %, while the upper limit may be from any one of about 54.5 wt. %, or about 55 wt. %.

### Method of Making Modified Legume Dough

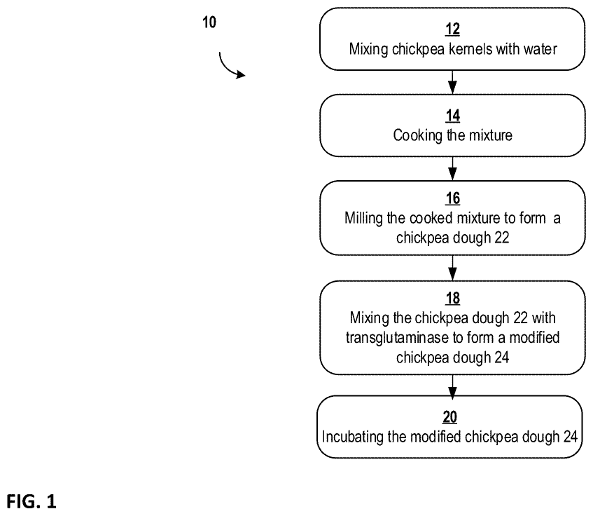

In one embodiment, the legume used to form the legume dough may be chickpea. FIG. 1 illustrates an exemplary method 10 for preparing a chickpea dough. The method 10 includes mixing 12 chickpea kernels with water to form a mixture and cooking 14 the mixture. The mixture may be cooked for about 45 min at about 145° F. The method 10 then includes the step of milling 16 the cooked chickpea kernels to form a chickpea dough 22.

Transglutaminase (TG) is added to the chickpea dough to form a modified chickpea dough 24. In some embodiments, a low oxidation TG may be used in the method 10 to form the modified chickpea dough 24. The TG (whether low oxidation TG or otherwise) may be pre-dispersed with water before adding to the chickpea dough 22 to form the modified chickpea dough 24. Typically, the amount of water used to pre-disperse the TG (or low oxidation TG) may be an amount suitable to wet or disperse the TG (or low oxidation TG) so that it can be effectively mixed with the chickpea dough. In some instances, the amount of water may be about 1 ml to about 10 ml. And, the water may suitably be deionized or purified water.

The amount of TG (or low oxidation TG) added to the chickpea dough 22 to form the modified chickpea dough 24 may range from about 0.3 wt. % to about 1.2 wt. % w/w, including any percentage or range comprised therein. In some embodiments, the amount of TG (or low oxidation TG) added to the chickpea dough 22 to form the modified chickpea dough 24 may range from about 0.3 wt. % to about 5 wt. % w/w, including any percentage or range comprised therein. The amount of TG (or low oxidation TG) added to the chickpea dough 22 to form the modified chickpea dough 24 may be about 0.3 wt. % to about 0.5 wt. %, about 0.5 wt. % to about 0.8 wt. %, about 0.8 wt. % to about 1.0 wt. %, about 1.0 wt. % to about 1.4 wt. %, about 1.4 wt. % to about 1.8 wt. %, about 1.8 wt. % to about 2.0 wt. %, about 2.0 wt. % to about 2.5 wt. %, about 2.5 wt. % to about 3.0 wt. %, about 3.0 wt. % to about 3.5 wt. %, about 3.5 wt. % to about 4.0 wt. %, about 4.0 wt. % to about 4.5 wt. %, or about 4.5 wt. % to about 5.0 wt. %, including any percentage or range comprised therein. The TG crosslinks the globulin and albumin found in the modified legume dough 24 to conserve protein content and reduced stickiness. As a result, TG (or low oxidation TG) modified legume dough 24 is more machinable and sheetable, particularly when compared to a legume dough not containing or treated with TG (or low oxidation TG).

After formation of the modified chickpea dough 24, it may be frozen, freeze-dried, ground to a powder, and/or incubated 20.

The incubation may be conducted in any known manner such as in a water bath. The modified dough 24 may be continuously stirred during incubation 20. In some embodiments, the modified chickpea dough 24 may be incubated for a period of time from about 5 minutes to about 15 minutes, about 15 minutes to about 25 minutes, from about 25 minutes to about 40 minutes, from about 40 minutes to about 60 minutes, from about 60 minutes to about 120 minutes, including any time period or range comprised therein. In some embodiments, the modified chickpea dough 24 may be incubated at a temperature of about 60° F. to about 70° F., about 70° F. to about 80° F., about 80° F. to about 100° F., from about 100° F. to about 120° F., from about 120° F. to about 130° F., or about 130° F. to about 140° F., including temperature or range comprised therein. In some instances, the incubation is conducted for a period of time of about 30 min at a temperature of about 104° F.

In some embodiments, the modified legume dough 24 may further comprise nixtamalized corn as described earlier. In some embodiments, the cooked chickpea and the nixtamalized corn may be blended, and then milled to create the modified legume dough 24. In other embodiments, the cooked chickpea can be milled first to form a first stock dough, and the nixtamalized corn can be milled separately to form a second stock dough. Then the first and the second stock doughs may be blended using to create the modified legume dough 24.

### Method of Making Snack Product

To create a tortilla-style chip, the modified legume dough 24 is discharged from the incubation apparatus, sheeted (passed through one or more rollers to form a sheet of dough), and then passed through a cutter to form individual cut pieces, which in some instances have the characteristic triangular-shape of tortilla-style chips. Thereafter, the individual pieces are dehydrated such as by baking or frying to produce the resulting tortilla-style chip snack product.

The sheets formed from the dough comprise a thickness of about 0.5 mm to about 3 mm, including any thickness or range comprised therein. Sheet thickness may be measured indirectly by weighing 10 cut raw chips. The sheets produced from the dough may comprise a weight of about 30 grams/10 raw chips to about 35 grams/10 raw chips, or about 35 grams/10 raw chips to about 40 grams/10 raw chips, including any weight or range comprised therein.

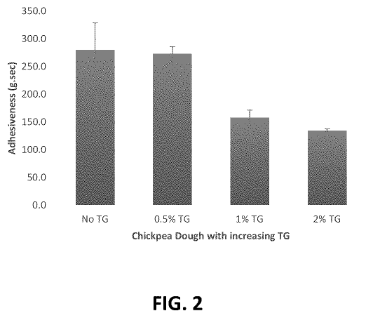

The dough adhesiveness of modified legume dough was measured using a TA.XTPIus Texture Analyzer (available from Texture Technologies Corp.) using a TA 25 probe (which is a 2 inch diameter aluminum cylinder that is 20 mm tall). As shown in FIG. 2, the absolute value of adhesiveness decreased with an increase in the amount of TG while the incubation time and temperature were kept constant. A decrease in the adhesiveness provides an indication of the stickiness or adhesion to the cutting roll, which is not desired. On the other hand, the dough must have some adhesiveness to enable desired sheet integrity as it is conveyed and passes through the cutting roll.

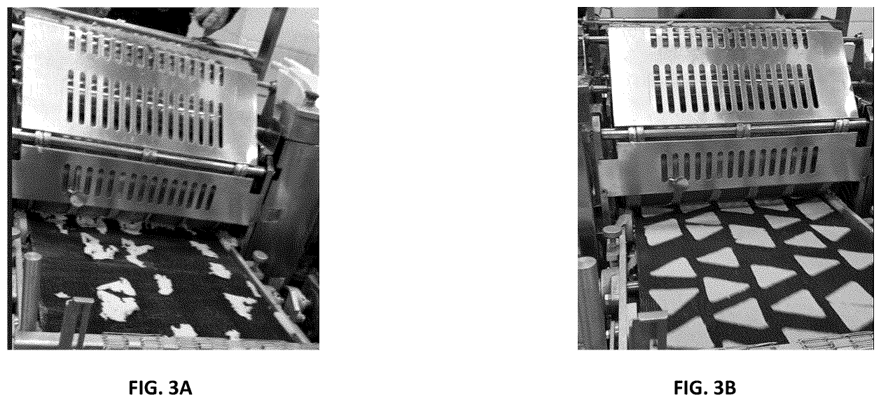

FIG. 3A illustrates an instance when shreds of dough exit the cutter as a result of a high level of dough stickiness, i.e., when the dough is not according to the described modified legume dough. On the other hand, FIG. 3B illustrates an instance when a dough according to the described modified legume dough exits the cutter as cleanly cut individual pieces. The clean cut chips are then toasted and conditioned to a moisture level of about 20-45%, and fried to a fried base chip moisture of about 0.9-1.5%. The fried chips are then seasoned using equipment known in the art and the resulting seasoned chips can be packed in a format suitable for their commercialization.

A semi-quantitative sheeting score was developed to evaluate the efficiency of dough transfer to the cutting roll, the release of the dough from the cutting roll onto the discharge conveyor, the level of dough shrinkage, and the cleanness of the cutting of the sheeted dough. The dough is passed through a sheeting unit operation comprising at least three sets of rollers: two symmetric (front and back) rollers that create the sheet and a third roll—the cutting roll—adjacent the two symmetric rolls that cuts the sheet into the desired chip shapes. The front and back rollers move concentrically, so the dough moves through a gap between the two rollers moving the dough to the front roller. This is typically achieved by applying a speed differential, where the front roller moves faster than the back roller, thus moving the dough to the front roller. When the dough is very sticky, it deposits on the rollers and the dough is not able to be moved downstream as a whole sheet.

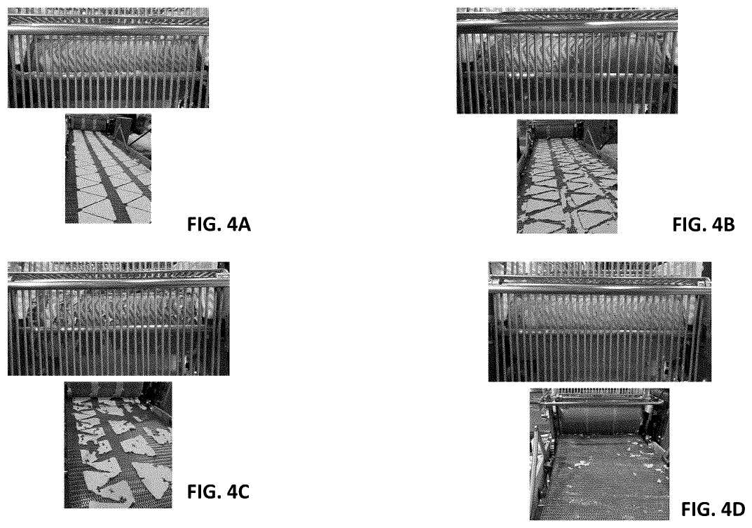

A score in the range from 4 to 1 is assigned based on the above criteria. A sheeting score of 1 indicates that about 0% of the dough sticks to the cutting roll, which indicates that the dough is entirely or nearly entirely released from the sheeting unit operation, as shown in FIG. 4A. In addition, in this instance, it will be appreciated that the cut dough exhibits the desired triangular shape individual pieces without any extraneous dough pieces.

A score of 2 indicates that the sheet exhibits some dimensional shrinkage from a cutting mold in the direction parallel to the sheeting direction. A sheeting score of 2 further indicates that about 20% of the dough sticks to the cutting roll. The individual cut pieces may or may not be cleanly cut and shaped, as seen in FIG. 4B.

A sheeting score of 3 indicates that about 20% to about 50% of the dough sticks to the cutting roll. The individual cut pieces may not be cleanly cut and shaped, as seen in FIG. 4C.

A sheeting score of 4 indicates that nearly all or all of the dough sticks to the cutting roll and cannot be cut to form individual cut pieces, as seen in FIG. 4D.

As noted above, after the individual pieces are formed, they may be dehydrated by baking or frying. When fried, the pieces may be fried in any suitable oil such as corn, canola, safflower, sunflower, and the like. General frying conditions are known to those skilled in the art and need not be elaborated.

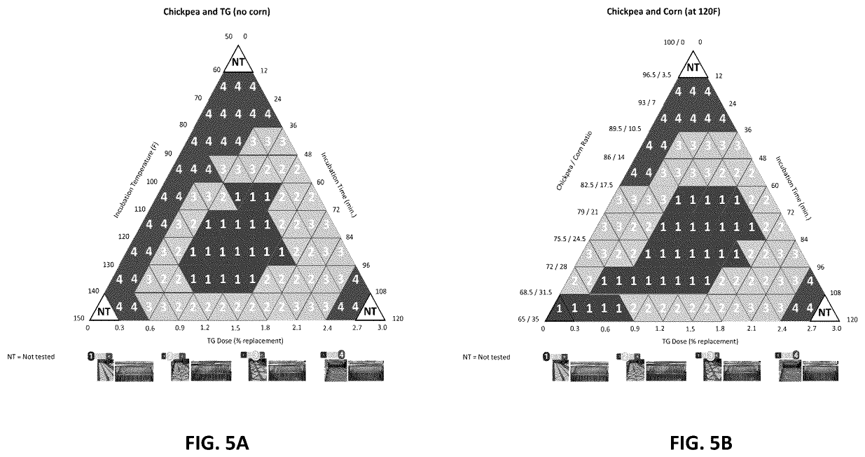

The impact on the sheetability of the modified legume dough as a result of adding different amounts of TG is shown in FIGS. 5A and 5B. In particular, FIG. 5A shows the sheeting score at varying incubation temperatures and varying TG amounts used to form a modified legume dough that contained all chickpea and no corn. FIG. 5B shows the sheeting score for a modified legume dough comprising both corn and chickpea, with varying amounts shown along one axis and with varying incubation temperatures and varying TG amounts along the other axes.

After frying, the snack product may be seasoned by application of one or more seasonings or flavorings applied to the surfaces of the individual snack product pieces in a known manner. It will be appreciated that any number of seasoning or flavoring particles or compositions may be applied. By way of example without intending to limit the scope of this disclosure, the additional seasoning or flavoring, may comprise sodium chloride, table salt, kosher salt, sea salt, pepper, paprika, dill, cinnamon, sugar, cardamom, ginger, mustard, parsley, sage, thyme, ranch, barbeque, cheese, vinegar, honey, sour cream, onion, jalapeno, chile limon, limon, dill pickle, adobadas, and any combination thereof.

In some embodiments, seasoning or flavoring may be added in minor amounts of no more than an average amount of about 10 wt. % per individual raw chip, depending on the type of flavor profile desired. In some embodiments, the seasoning or flavoring may be present on the snack product in an amount ranging from about 1 wt. % to about 10 wt. %, or about 1 wt. %, 2 wt. %, 3 wt. %, 4 wt. %, 5 wt. %, 6 wt. %, 7 wt. %, 8 wt. %, 9 wt. %, or about 10 wt. %. In some embodiments, the seasoning or flavoring may be present on the snack product in a range where the lower limit may be from any one of about 1 wt. %, 2 wt. %, 3 wt. %, 4 wt. %, 5 wt. %, 6 wt. %, 7 wt. %, 8 wt. %, 9 wt. %, while the upper limit may be from anyone of about 2 wt. %, 3 wt. %, 4 wt. %, 5 wt. %, 6 wt. %, 7 wt. %, 8 wt. %, 9 wt. %, 6 wt. %, 7 wt. %, 8 wt. %, 9 wt. %, or about 10 wt. %. In some embodiments, the seasoning or flavoring present on the snack product may be more than 10% or less than 0%.

Thereafter, the snack product (whether seasoned or not) is packaged into a suitable package.

### Resulting Snack Product

In some aspects, the snack product comprises, consists essentially of, or consists of a legume, deactivated transglutaminase (TG), and in some instances nixtamalized corn, oil, one or more seasonings or flavorings, and water. In some embodiments, the snack product comprises, consists essentially of, or consists of a legume, deactivated low oxidation TG, and in some instances nixtamalized corn, oil, one or more seasonings or flavorings, and water. Deactivated TG or deactivated low oxidation TG is formed when the TG or low oxidation TG is exposed to a temperature above a threshold temperature of about 60° C. (140° F.), which causes the TG or the low oxidation TG to denature, consequently losing all catalytic activity and rendering it inactive. The modified legume dough used to make the snack product may comprise nixtamalized corn, legume, and TG. In some embodiments, the modified legume dough used to make the snack product may comprise nixtamalized corn, legume, and low oxidation TG. In some embodiments, the modified legume dough may further comprise flavoring, antioxidant, and/or other functional agent.

The legume may be present in the snack product in amounts ranging from about 20 wt. % to about 30 wt. %, about 30 wt. % to about 40 wt. %, about 40 wt. % to about 50 wt. %, about 50 wt. % to about 60 wt. %, about 60 wt. % to about 70 wt. %, about 70 wt. % to about 80 wt. %, about 80 wt. % to about 90 wt. %, or about 80 wt. % to about 99 wt. %, including any percentage or range comprised therein.

When present, the amount of nixtamalized corn in the snack product may be from about 1 wt. % to about 10 wt. %, about 10 wt. % to about 20 wt. %, about 20 wt. % to about 30 wt. %, or about 30 wt. % to about 40 wt. %, including any percentage or range comprised therein.

As noted above, in some embodiments, the TG (or low oxidation TG) in the snack product may be deactivated TG (or deactivated low oxidation TG). In some embodiments, the amount of deactivated TG (or deactivated low oxidation TG) in the snack product may be less than the amount of TG (or low oxidation TG) added to the modified legume dough used to make the snack product. The amount of deactivated TG (or low oxidation TG) in the snack product may be about 0.3 wt. % to about 0.5 wt. %, about 0.5 wt. % to about 0.8 wt. %, about 0.8 wt. % to about 1.0 wt. %, about 1.0 wt. % to about 1.4 wt. about 1.4 wt. % to about 1.8 wt. %, about 1.8 wt. % to about 2.0 wt. %, about 2.0 wt. % to about 2.5 wt. %, about 2.5 wt. % to about 3.0 wt. %, about 3.0 wt. % to about 3.5 wt. %, about 3.5 wt. % to about 4.0 wt. %, about 4.0 wt. % to about 4.5 wt. %, or about 4.5 wt. % to about 5.0 wt. %, including any percentage or range comprised therein.

The amount of oil in the snack product may be about 23 wt. % to about 25 wt. %, about 25 wt. % to about 27 wt. %, about 27 wt. % to about 29 wt. %, about 29 wt. % to about 31 wt. %, about 31 wt. % to about 32 wt. %, about 32 wt. % to about 34 wt. %, including any percentage or range comprised therein. In some embodiments, the snack product may comprise an oil pickup level of about 22 wt. % to about 23 wt. %, or from about 23 wt. % to about 24 wt. %, including any percentage or range comprised therein. In some embodiments, the snack product may comprise an oil pickup level of less than about 30%.

The amount of seasonings or flavorings in the snack product may be about 6 wt. % to about 7 wt. %, about 7 wt. % to about 8 wt. %, about 8 wt. % to about 9 wt. %, about 9 wt. % to about 10 wt. %, about 10 wt. % to about 11 wt. %, or about 11 wt. % to about 12 wt. %, including any percentage or range comprised therein. The amount of moisture in the snack product may be about 0.5 wt. % to about 0.7 wt. %, about 0.7 wt. % to about 0.9 wt. %, about 0.9 wt. % to about 1.1 wt. %, or about 1.1 wt. % to about 1.3 wt. %, about 1.3 wt. % to about 1.5 wt. %, about 1.5 wt. % to about 01.7 wt. %, about 1.7 wt. % to about 2.0 wt. %, including any percentage or range comprised therein.

In some embodiments, the snack product may contain about 1 wt. % to about 5 wt. %, about 5 wt. % to about 8 wt. %, about 8 wt. % to about 10 wt. %, about 10 wt. % to about 13 wt. %, about 13 wt. % to about 15 wt. %, about 15% to about 20%, about 20% to about 30%, about 30% to about 40%, about 40% to about 50%, about 50% to about 60%, about 60% to about 70%, about 70% to about 80%, or about 80% to about 90% of protein, depending upon the legume used in the modified legume dough. For example, chickpeas comprise about 20 wt. % protein. Therefore chickpea can provide a total of 20 grams protein per 100 g of chickpeas.

### EXAMPLES

### Example 1: Effect of Transglutaminase (TG) on Legume Properties

The effect of transglutaminase (TG) on chickpea properties was evaluated at 145° F. Table 1 shows the solubility (g/100 g dry flour) and water holding capacity (WHC) of chickpea dough after TG treatment. Specifically, Table 1 shows the amount of soluble matter (grams) per 100 gram freeze dried dough. It was observed that the addition of about 0.5 wt. % TG treatment greatly reduced the protein solubility of cooked chickpea dough, e.g., from about 22.59 to about 19.23 g/100 g dough (p<0.05) and changed the sheeting score from 4 to 3. For reference, the solubility in raw corn dough is about 19.8 g/100 g dough and the protein solubility in dough made with corn cooked at 205° F. is about 9.38 g/100 g dough.

Increasing the amount of TG from 0.5 wt. % to 1.0 wt. % directionally enhanced the reduction of chickpea dough solubility (19.23 vs. 18.64 g/100 g dough) (p>0.05) and improved the sheeting score to 2. The WHC of the chickpea dough also significantly increased by the treatment of TG, changing from 1.060 to 1.228 g/g, when treated with 1.0 wt. % TG (Table 1). TG is an enzyme generally used to covalently cross-link the amino group on lysine residues and the carboxyamide group on glutamine residues, which result in a modification of protein structure and functionality. Consequently, both the solubility reduction and the WHC increase of chickpea dough after TG treatment were possibly related to protein aggregation and polymerization caused by TG.

Pasting properties as related to viscosity of chickpea dough after TG treatment were analyzed. As shown in Table 2, treated whole chickpea dough control exhibited similar viscosity compared to the untreated whole chickpea dough (1304.5 vs. 1399.5 cP for peak viscosity, 1811.0 vs. 1859.5 cP for final viscosity), suggesting that incubation in water bath for 30 min at 40° C. did not induce significant starch gelatinization. However, TG treatments greatly increased the peak and final viscosities of chickpea dough, with the lower concentration of TG being more capable of increasing dough viscosity and cohesiveness than the higher dosage (1637.5 vs. 1507 cP for the peak viscosity of 0.5 wt. % TG and 1.0 wt. % TG, and 2120.5 vs. 1827.5 cP for the final viscosity of 0.5 wt. % TG and 1.0 wt. % TG, respectively). TG was able to induce protein cross-linking and enhance protein network, which appears to increase the dough's ability to retain water and to resist shear force and deformation. Such enhanced protein network may also contribute to the increased peak and final viscosities of chickpea dough when treated with TG at an appropriate concentration.

TABLE 1
Solubility and water holding capacity (WHC) of whole chickpea
dough from transglutaminase (TG) modification

| | | Solubility of | |
|---|---|---|---|
| | | as-is pH | WHC |
| | | (g/100 g | (g H2O/g |
| | Original pH | dough) | dough) |
| Treated whole | 6.52 ± 0.00 c | 22.59 ± 0.22 a | 1.060 ± 0.009 c |
| chickpea dough | | | |
| control - 145° F. * | | | |
| Whole chickpea | 6.59 ± 0.01 b | 19.23 ± 0.89 b | 1.118 ± 0.008 b |
| dough - 145° F.- | | | |
| 0.5 wt. % TG | | | |
| Whole chickpea | 6.62 ± 0.01 a | 18.64 ± 0.91 b | 1.228 ± 0.005 a |
| dough - 145° F.- | | | |
| 1 wt. % TG | | | |

Note:
Results are expressed as mean ± SD (n = 3). Different letters within the same column indicate significant difference (p < 0.05).
* Treated whole chickpea dough control-145° F. indicated that after water cooking at 145° F., chickpea kernels were milled using a stone mill and underwent further water bath treatment (40° C., 30 min) but without TG addition.

TABLE 2
RVA pasting property of chickpea dough from TG modification

| | | | | | | Peak | Pasting |
|---|---|---|---|---|---|---|---|
| | Viscosity (cP) | | | | | Time | Temp |
| | Peak | Trough | Breakdown | Final | Setback | (min) | (° C.) |
| Untreated | 1399.5 ± | 1258 ± | 141.5 ± | 1859.5 ± | 601.5 ± | 7 ± | 79.575 ± |
| chickpea | 17.68 c | 24.04 c | 6.36 b | 10.61 b | 13.44 b | 0.00 a | 0.67 a |
| dough-145 F. * | | | | | | | |
| Treated | 1304.5 ± | 1125.5 ± | 179.0 ± | 1811.0 ± | 685.5 ± | 7 ± | 79.85 ± |
| chickpea | 6.36 d | 0.71 d | 5.66 a | 16.97 c | 16.26 a | 0.00 a | 0.00 a |
| dough | | | | | | | |
| control-145 F. | | | | | | | |
| Chickpea | 1637.5 ± | 1526 ± | 111.5 ± | 2120.5 ± | 594.5 ± | 7 ± | 77.475 ± |
| dough-145 F.- | 2.12 a | 4.24 a | 2.12 c | 0.71 a | 4.95 b | 0.00 a | 0.04 b |
| 0.5% TG | | | | | | | |
| Chickpea | 1507 ± | 1478 ± | 29 ± | 1827.5 ± | 349.5 ± | 6.67 ± | 77.45 ± |
| dough-145 F.- | 5.66 b | 2.83 b | 2.83 d | 6.36 c | 9.19 c | 0.09 b | 0.00 b |
| 1% TG | | | | | | | |

Note:
Result are expressed as mean ± SD (n = 2). Different letters within the same column indicate significant difference (p < 0.05).
* Untreated whole chickpea dough-145 F. indicated that after water cooking at 145° F., chickpea kernels were milled to dough using a stone mill without further water bath treatment;
** Treated whole chickpea dough control-145 F. indicated that after water cooking at 145° F., chickpea kernels were milled to dough using a stone mill and underwent further water bath treatment (40° C., 30 min) but without TG addition.

### Example 2: Hardness of Snack Product

Using a calibrated texture analyzer instrument such as a TA.TXPlus Connect or a TA.XTPlus100 (Texture Technologies Corp. and Stable Micro Systems, Ltd. Hamilton, MA) a whole chip is placed on top of a ring-like fixture compatible with the TA-8¼ ball probe (Texture Technologies Corp. and Stable Micro Systems, Ltd. Hamilton, MA) on a testing platform. Using the appropriate computer software such as Texture Exponent (Texture Technologies Corp. and Stable Micro Systems, Ltd. Hamilton, MA) with the parameters listed in Table 3, the probe was brought into contact with the chip using the pre-test speed shown in Table 3. At contact, the speed changes to the test speed, and moved a distance of 4 mm (as shown in Table 3). The chip broke during this distance, and the probe was then removed at the post speed shown in Table 3. The hardness is the peak force detected by the instrument as it pierces the chip. It is automatically calculated by the instrument. The described snack product exhibited a hardness from about 650 g force to about 750 g force.

TABLE 3
Puncture Method Test
TA-8 ¼″ Ball Probe

| | | |
|---|---|---|
| Pre-Test Speed: | 2 | mm/sec |
| Test Speed: | 0.5 | mm/sec |
| Post Test Speed: | 10 | mm/sec |
| Distance: | 4 | mm |
| Trigger: | 10 | g |

A bulk crunchiness test of snack products was measured using a calibrated texture analyzer instrument such as a TA.TXPIus Connect or a TA.XTPIus100 (Texture Technologies Corp. and Stable Micro Systems, Ltd. Hamilton, MA). A minimum of 5 whole chips were placed inside a 1.1″ crunchiness fixture and centered on the testing platform. A TA-25C probe (Texture Technologies Corp. and Stable Micro Systems, Ltd. Hamilton, MA) was used on the testing platform. Using the appropriate computer software such as Texture Exponent (Texture Technologies Corp. and Stable Micro Systems, Ltd. Hamilton, MA) with the parameters for the test, like those listed in Table 4 for chip crunchiness, the probe was brought to contact with the chip. At contact, the speed changed to a test speed, and moved a distance of 50 mm (as shown in Table 4). The chips broke during this distance, and the probe was then removed at the post speed shown in Table 4. The crunchiness is a property manifested by a tendency when the chips are subjected to an applied force to yield suddenly with a characteristic sound. It is a common property of low-moisture snacks like potato and tortilla chips. Their textural plots are usually associated with sharp triangular curves displaying an obvious break point (when tested individually) or a “jagged” multi-peak curve when tested in bulk. The number of peaks produced are the result of the fracture events that have occurred during the test. The average drop off can be determined by counting the number of peaks and measuring linear distance. Crunchiness takes into account the number of curves, the magnitude of the drop from a peak to a trough and the linear distance. All these parameters are detected and captured by the instrument as the instrument pierces the chip. The software can be configured to automatically calculate this parameter by the instrument. The snack chips made according to the described methods exhibited a “crunchiness” value of about 9000 g force to about 12000 g force.

TABLE 4
Bulk Crunchiness Test
TA-25 C 1.1″ Crunchiness Fixture
70 mm Start Height; 20 g

| Test Speed: | 2 | mm/sec |
|---|---|---|
| Post Test Speed: | 10 | mm/sec |
| Distance: | 50 | mm |
| Trigger: | Button | |
| Test Speed: | 2 | mm/sec |

Where an embodiment is described as comprising some element or group of elements, additional embodiments can consist essentially of or consist of the element or group of elements. Also, although the open-ended term “comprises” is generally used in this description, additional embodiments can be formed by substituting the terms “consisting essentially of” or “consisting of.”

Additionally, when a range for a particular variable is given for an embodiment, an additional embodiment can be created using a subrange or individual values that are contained within the range. Moreover, when a value, values, a range, or ranges for a particular variable are given for one or more embodiments, an additional embodiment can be created by forming a new range whose endpoints are selected from any expressly listed value, any value between expressly listed values, and any value contained in a listed range. For example, if the application were to disclose an embodiment in which a variable is 1 and a second embodiment in which the variable is 3-5, a third embodiment can be created in which the variable is 1.31-4.23. Similarly, a fourth embodiment can be created in which the variable is 1-5.

While the invention has been described in terms of specific or particular embodiments, it should be apparent that alternatives could be adopted by one skilled in the art. In addition, the invention encompasses additional or alternative embodiments in which one or more features or aspects of a particular embodiment could be eliminated or two or more features or aspects of different disclosed embodiments could be combined. Accordingly, it should be understood that the invention is not necessarily limited to any embodiment described herein or illustrated in the drawings. It should also be understood that the purpose of the above detailed description and the phraseology and terminology employed therein is to describe the illustrated embodiments, and not necessarily to serve as limitations to the scope of the invention. Finally, while the appended claims recite certain aspects believed to be associated with the invention, they do not necessarily serve as limitations to the scope of the invention.

1. A dough comprising about 60 wt. % to about 100 wt. % legume and transglutaminase.

2. The dough of claim 1, wherein the legume is one or more of peas, grass peas, green peas, black-eyed peas, broad beans, chickpeas, lentils, lupins, mesquite, carob, tamarind, alfalfa, clover, soybeans, lima beans, black beans, kidney beans, navy beans, butter beans, adzuki beans, peanuts, pinto beans or mixtures thereof.

3. The dough of claim 1 wherein the legume is in the form of whole seeds, whole cooked seeds, partially or fully dehulled/decorticated seeds, partially or fully dehulled/decorticated cooked seeds, partially or fully de-oiled seeds, raw whole grits, raw partially or fully dehulled/decorticated grits, partially or fully de-oiled grits, raw whole flours, raw partially or fully dehulled/decorticated flours, raw partially or fully de-oiled flours, raw protein fractions, readily soluble legume protein, and mixtures thereof.

4. The dough of claim 1 wherein the transglutaminase is a low oxidation transglutaminase.

5. The dough of claim 1 comprising about 0.5 wt. % to about 5 wt. % transglutaminase.

6. The dough of claim 1 in a form of a sheet.

7. The dough of claim 6, wherein the sheet comprises a thickness of about 0.5 mm to about 3.0 mm.

8. The dough of claim 1, wherein the dough has about 20 grams of soluble matter per 100 grams of the dough.

9. A snack product comprising about 40 wt. % to about 100 wt. % legume, deactivated transglutaminase, and less than about 30 wt. % oil.

10. The snack product of claim 8, wherein the legume is chickpea, lentil, or pinto bean.

11. The snack product of claim 8, wherein the snack product comprises about 23 wt. % oil.

12. The snack product of claim 10, further comprises about 10% seasonings.

13. A method of preparing a modified dough comprising:
providing a legume dough that comprises between about 60 wt. % to about 100 wt. % legume;
adding about 0.05 wt. % to about 5 wt. % of transglutaminase to the legume dough; and
holding the transglutaminase-containing legume dough at a temperature between about 100° F. to about 140° F. for between about 5 minutes to about 120 minutes to make the modified dough.

14. The method of claim 13, wherein the legume is chickpea, lentil, or pinto bean.

15. The method of claim 13 further comprising sheeting the modified dough, cutting the sheeted modified dough into individual pieces, and frying the individual pieces to form snack product, wherein the fried pieces have an oil intake of less than about 30 wt.

16. The method of claim 15, wherein the snack product comprises about 10 wt. % of seasonings.

17. The method of claim 13, wherein the method conserves about 10% to about 100% original protein in the legume used to make the legume dough.

18. The method of claim 13, wherein the legume dough is prepared by mixing the legume with water at a temperature between about 65° F. and about 212° F.
